(19) United States
(12) Patent Application Publication
(10) Pub. No.: US 2024/0181185 A1
(43) Pub. Date: Jun. 6, 2024

(54) SYSTEMS AND METHODS FOR CHARACTERIZING A URSER INTERFACE OR A VENT USING ACOUSTIC DATA ASSOCIATED WITH THE VENT

(71) Applicant: ResMed Sensor Technologies Limited, Dublin (IE)

(72) Inventors: Niall Andrew FOX, Dublin (IE); Roxana TIRON, Dublin (IE)

(21) Appl. No.: 18/554,262

(22) PCT Filed: Apr. 8, 2022
(86) PCT No.: PCT/IB2022/053332

§ 371 Date: Oct. 6, 2023

Related U.S. Application Data
(60) Provisional application No. 63/176,097, filed on Apr. 16, 2021.

(51) Int. Cl. A61M 16/00; A61M 16/20

(52) U.S. Cl. CPC A61M 16/0069; A61M 16/026; A61M 16/204; A61M 16/205; A61M 2205/3375

(57) ABSTRACT

According to some implementations of the present disclosure, a method includes receiving acoustic data associated with airflow caused by operation of a respiratory therapy system, which is configured to supply pressurized air to a user. The respiratory therapy system includes a user interface and a vent. The method also includes determining, based at least in part on a portion of the received acoustic data, an acoustic signature associated with the vent. The method also includes characterizing, based at least in part on the acoustic signature associated with the vent, the user interface, the vent, or both.

### TECHNICAL FIELD

The present disclosure relates generally to systems and methods for characterizing a user interface and/or a vent of the user interface, and more particularly, to systems and methods for characterizing a user interface and/or a vent of the user interface using acoustic data associated with the vent.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA) and Central Sleep Apnea (CSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using respiratory therapy systems.

Each respiratory system generally has a respiratory therapy device connected to a user interface (e.g., a mask) via a conduit and optionally a connector. The user wears the user interface and is supplied a flow of pressurized air from the respiratory therapy device via the conduit. The user interface generally is a specific category and type of user interface for the user, such as direct or indirect connections for the category of user interface, and full face mask, a partial face mask, nasal mask, or nasal pillows for the type of user interface. In addition to the specific category and type, the user interface generally is a specific model made by a specific manufacturer. For various reasons, such as ensuring the user is using the correct user interface, it can be beneficial for the respiratory system to know the specific category and type, and optionally specific model, of the user interface worn by the user.

Thus, it is advantageous to know the user interface of a respiratory therapy system for providing improved control of therapy delivered to the user. For instance, it may be advantageous to know the user interface in order to accurately measure or estimate treatment parameters, such as pressure in the user interface and vent flow. Accordingly, knowledge of what user interface is being used can enhance therapy. Although some respiratory therapy devices may include a menu system that allows a user to enter the type of user interface being used (e.g., by type, model, manufacturer, etc.), the user may enter incorrect or incomplete information. As such, it may be advantageous to determine the user interface independently of user input.

In addition, vents on the user interface or on a connector to the user interface can deteriorate over time, become blocked or occluded due to a buildup of unwanted material (e.g., saliva, mucus, skin cells, bedding fibers, debris from the user interface), or become temporarily blocked or occluded (e.g. against bedding or a pillow). A deteriorated and/or occluded vent can cause the vent-flow performance of the user interface to deviate from the nominal performance, which may impact therapy comfort or therapy accuracy. The deteriorated and/or the occluded vent can also lead to a buildup of CO₂, which in turn may result in inefficient therapy, additional noise, patient discomfort, or even danger to the user. Thus, when the vent is deteriorated or occluded, it can negatively impact therapy. As a result, some users will discontinue use of the respiratory therapy system because of the discomfort and/or inaccurate therapy caused by the deteriorated or occluded vent.

The present disclosure is directed to solving these and other problems.

### SUMMARY

According to some implementations of the present disclosure, a method includes receiving acoustic data associated with airflow caused by operation of a respiratory therapy system, which is configured to supply pressurized air to a user. The respiratory therapy system includes a user interface and a vent. The method also includes determining, based at least in part on a portion of the received acoustic data, an acoustic signature associated with the vent. The method also includes characterizing, based at least in part on the acoustic signature associated with the vent, the user interface, the vent, or both.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

According to some implementations of the present disclosure, a system for characterizing a user interface and/or a vent of a respiratory therapy system includes a control system configured to implement any one of the methods disclosed herein.

According to some implementations of the present disclosure, a computer program product includes instructions which, when executed by a computer, cause the computer to carry out any one of the methods disclosed herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of one category of user interfaces, according to some implementations of the present disclosure.

FIG. 3B is an exploded view of the user interface of FIG. 3A, according to some implementations of the present disclosure.

FIG. 4A is a perspective view of another category of user interfaces, according to some implementations of the present disclosure.

FIG. 4B is an exploded view of the user interface of FIG. 4A, according to some implementations of the present disclosure.

FIG. 5A is a perspective view of another category of user interfaces, according to some implementations of the present disclosure.

FIG. 5B is an exploded view of the user interface of FIG. 5A, according to some implementations of the present disclosure.

FIG. 6 is a rear perspective view of a respiratory therapy device of the system of FIG. 6, according to some implementations of the present disclosure;

FIG. 7 is a process flow diagram for a method for characterizing a user interface or a vent of the user interface, according to some implementations of the present disclosure;

FIG. 8 illustrates patient flow and user interface pressure over a period of 2,000 seconds during pressure ramp-up, according to some implementations of the present disclosure;

FIG. 9 illustrates the log audio spectra versus frequency during the pressure ramp-up of FIG. 8, according to some implementations of the present disclosure;

FIG. 10A illustrates an acoustic signature for a first user interface (AirFit™ F10 model) across the frequency between 0 to 10 kHz, according to some implementations of the present disclosure;

FIG. 10B illustrates an acoustic signature for a second user interface (AirFit™ F20 model) across the frequency between 0 to 10 kHz, according to some implementations of the present disclosure;

FIG. 10C illustrates an acoustic signature for a third user interface (AirFit™ N30 model) across the frequency between 0 to 10 kHz, according to some implementations of the present disclosure;

FIG. 10D illustrates an acoustic signature for a fourth user interface (AirFit™ N30i model) across the frequency between 0 to 10 kHz, according to some implementations of the present disclosure;

FIG. 10E illustrates an acoustic signature for a fifth user interface (Brevida™ model (Fisher & Paykel)) across the frequency between 0 to 10 kHz, according to some implementations of the present disclosure;

FIG. 10F illustrates an acoustic signature for a sixth user interface (DreamWear™ FullFace model (Philips Respironics)) across the frequency between 0 to 10 kHz, according to some implementations of the present disclosure;

FIG. 10G illustrates an acoustic signature for a seventh user interface (Eson2™ Nasal model (Fisher & Paykel)) across the frequency between 0 to 10 kHz, according to some implementations of the present disclosure;

FIG. 10H illustrates an acoustic signature for an eighth user interface (Simplus™ model (Fisher & Paykel)) across the frequency between 0 to 10 kHz, according to some implementations of the present disclosure;

FIG. 10I illustrates an acoustic signature for a ninth user interface (AirFit™ F30 model) across the frequency between 0 to 10 kHz, according to some implementations of the present disclosure;

FIG. 10J illustrates an acoustic signature for a tenth user interface (AirFit™ F30i model) across the frequency between 0 to 10 kHz, to some implementations of the present disclosure;

FIG. 10K illustrates an acoustic signature for an eleventh user interface (AirFit™ P10 model) across the frequency between 0 to 10 kHz, according to some implementations of the present disclosure;

FIG. 10L illustrates an acoustic signature for a twelfth user interface (AirFit™ P30i model) across the frequency between 0 to 10 kHz, according to some implementations of the present disclosure;

FIG. 10M illustrates an acoustic signature for a thirteenth user interface (DreamWear™ Nasal model (Philips Respironics)) across the frequency between 0 to 10 kHz, according to some implementations of the present disclosure;

FIG. 10N illustrates an acoustic signature for a fourteenth user interface (DreamWear™ Pillows model (Philips Respironics)) across the frequency between 0 to 10 kHz, according to some implementations of the present disclosure;

FIG. 10O illustrates an acoustic signature for a fifteenth user interface (Vitera™ model (Fisher & Paykel)) across the frequency between 0 to 10 kHz, according to some implementations of the present disclosure;

FIG. 10P illustrates an acoustic signature for a sixteenth user interface (Wisp™ Nasal model (Philips Respironics)) across the frequency between 0 to 10 kHz, according to some implementations of the present disclosure;

FIG. 10Q illustrates an acoustic signature for a seventeenth user interface (AirFit™ N20 model) across the frequency between 0 to 10 kHz, according to some implementations of the present disclosure;

FIG. 10R illustrates an acoustic signature for an eighteenth user interface (DreamWisp™ model (Philips Respironics)) across the frequency between 0 to 10 kHz, according to some implementations of the present disclosure;

FIG. 10S illustrates an acoustic signature for a nineteenth user interface (AmaraView™ model (Philips Respironics)) across the frequency between 0 to 10 kHz, according to some implementations of the present disclosure;

FIG. 11A illustrates the spectra acoustic signature versus frequency for open vents and partially occluded vents, according to some implementations of the present disclosure;

FIG. 11B illustrates the spectra acoustic signature versus frequency for open vents and fully occluded vents, according to some implementations of the present disclosure;

FIG. 11C illustrates the spectra acoustic signature versus frequency for open vents and completely occluded vents (including anti-asphyxia valve), according to some implementations of the present disclosure;

FIG. 12A illustrates the cepstra acoustic signature versus frequency for open vents and partially occluded vents, according to some implementations of the present disclosure;

FIG. 12B illustrates the cepstra acoustic signature versus frequency for open vents and fully occluded vents, according to some implementations of the present disclosure; and

FIG. 12C illustrates the cepstra acoustic signature versus frequency for open vents and completely occluded vents (including anti-asphyxia valve), according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas (e.g., mixed apneas and hypopneas), Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for 10 seconds or longer leading to arousal from sleep and which does not fulfill the criteria for an apnea or hypopnea event. In 1999, the AASM Task Force defined RERAs as “a sequence of breaths characterized by increasing respiratory effort leading to an arousal from sleep, but which does not meet criteria for an apnea or hypopnea. These events must fulfil both of the following criteria: 1. pattern of progressively more negative esophageal pressure, terminated by a sudden change in pressure to a less negative level and an arousal; 2. the event lasts 10 seconds or longer. In 2000, the study “Non-Invasive Detection of Respiratory Effort-Related Arousals (RERAs) by a Nasal Cannula/Pressure Transducer System” done at NYU School of Medicine and published in Sleep, vol. 23, No. 6, pp. 763-771, demonstrated that a Nasal Cannula/Pressure Transducer System was adequate and reliable in the detection of RERAs. A RERA detector may be based on a real flow signal derived from a respiratory therapy (e.g., PAP) device. For example, a flow limitation measure may be determined based on a flow signal. A measure of arousal may then be derived as a function of the flow limitation measure and a measure of sudden increase in ventilation. One such method is described in WO 2008/138040, assigned to ResMed Ltd., the disclosure of which is hereby incorporated herein by reference.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

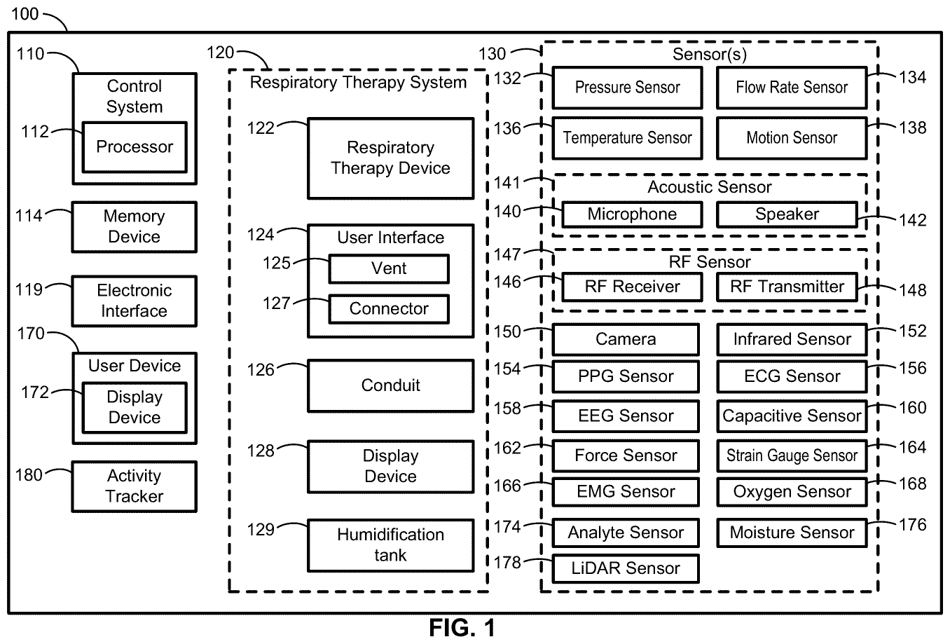

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120, and an activity tracker 180.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is illustrated in FIG. 1, the control system 110 can include any number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, a portion (e.g., a housing) of the respiratory therapy system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of a respiratory therapy device 122 of the respiratory therapy system 120, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a Wi-Fi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy (RPT) device 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 122 can also deliver pressurized air at a predetermined flow rate between, for example, about −20 L/min and about 150 L/min, while maintaining a positive pressure (relative to the ambient pressure).

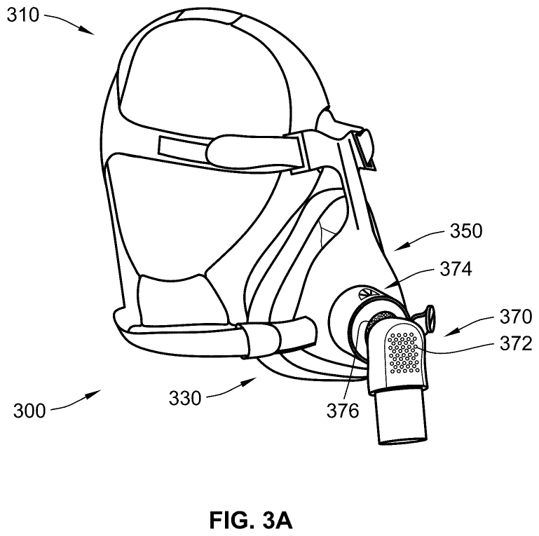

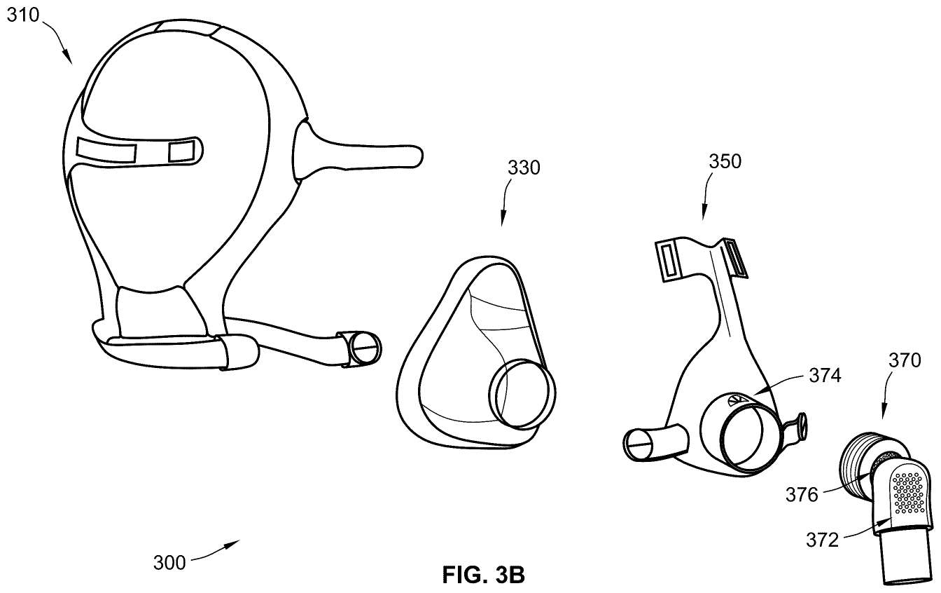

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O. In some implementations, the user interface 124 may include a connector 127 and one or more vents 125, which are described in more detail with reference to FIGS. 3A-3B, 4A-4B, and 5A-5B. In some implementations, the connector 127 is distinct from, but couplable to, the user interface 124 (and/or conduit 126).

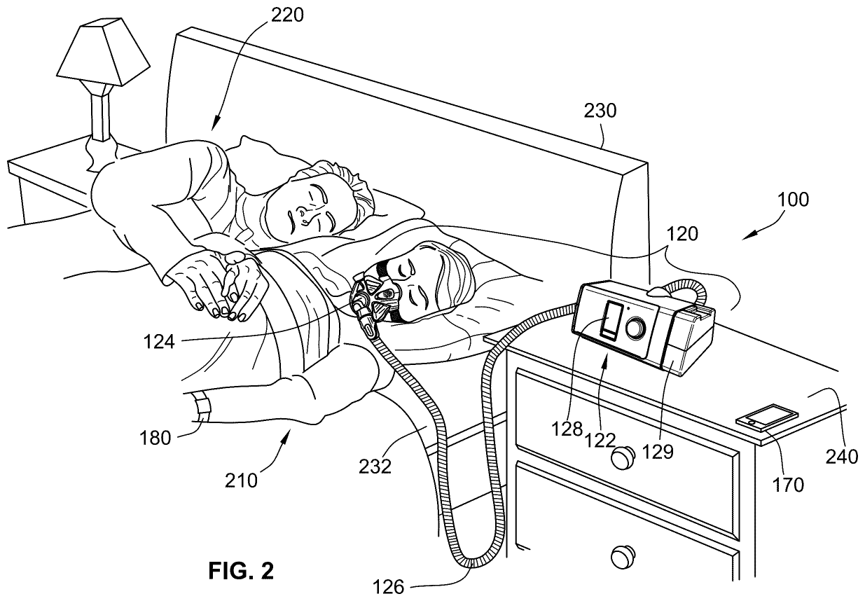

As shown in FIG. 2, in some implementations, the user interface 124 is a facial mask (e.g., a full face mask) that covers the nose and mouth of the user. Alternatively, the user interface 124 can be a nasal mask that provides air to the nose of the user or a nasal pillow mask that delivers air directly to the nostrils of the user. The user interface 124 can include a plurality of straps forming, for example, a headgear for aiding in positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the teeth of the user, a mandibular repositioning device, etc.).

FIGS. 3A and 3B illustrate a perspective view and an exploded view, respectively, of one implementation of a directly connected user interface (“direct category” user interfaces), according to aspects of the present disclosure. The direct category of a user interface 300 generally includes a cushion 330 and a frame 350 that define a volume of space around the mouth and/or nose of the user. When in use, the volume of space receives pressurized air for passage into the user's airways. In some embodiments, the cushion 330 and frame 350 of the user interface 300 form a unitary component of the user interface. The user interface 300 assembly may further be considered to comprise a headgear 310, which in the case of the user interface 300 is generally a strap assembly, and optionally a connector 370. The headgear 310 is configured to be positioned generally about at least a portion of a user's head when the user wears the user interface 300. The headgear 310 can be coupled to the frame 350 and positioned on the user's head such that the user's head is positioned between the headgear 310 and the frame 350. The cushion 330 is positioned between the user's face and the frame 350 to form a seal on the user's face. The optional connector 370 is configured to couple to the frame 350 and/or cushion 330 at one end and to a conduit of a respiratory therapy device (not shown). The pressurized air can flow directly from the conduit of the respiratory therapy system into the volume of space defined by the cushion 330 (or cushion 330 and frame 350) of the user interface 300 through the connector 370). From the user interface 300, the pressurized air reaches the user's airway through the user's mouth, nose, or both. Alternatively, where the user interface 300 does not include the connector 370, the conduit of the respiratory therapy system can connect directly to the cushion 330 and/or the frame 350.

In some implementations, the connector 370 may include a plurality of vents 372 located on the main body of the connector 370 itself and/or a plurality of vents 376 (“diffuser vents”) in proximity to the frame 350, for permitting the escape of carbon dioxide (CO₂) and other gases exhaled by the user when the respiratory therapy device is active. In some implementations, the frame 350 may include at least one plus anti-asphyxia valve (AAV) 374, which allows CO₂ and other gases exhaled by the user to escape in the event that the vents (e.g., the vents 372 or 376) fail when the respiratory therapy device is active. In general, AAVs (e.g., the AAV 374) are always present for full face masks (as a safety feature), however, the diffuser vents and vents placed on mask or connector (usually an array of orifices in the mask material itself or a mesh made of some sort of fabric, in many cases replaceable) are not both present (e.g., some masks might have only the diffuser vents such as the plurality of vents 376, other masks might have only the plurality of vents 372 on the connector itself).

For indirectly connected user interfaces (“indirect category” user interfaces), and as will be described in greater detail below, the conduit of the respiratory therapy system connects indirectly with the cushion and/or frame of the user interface. Another element of the user interface—besides any connector—is between the conduit of the respiratory therapy system and the cushion and/or frame. This additional element delivers the pressurized air to the volume of space formed between the cushion (or frame, or cushion and frame) of the user interface and the user's face, from the conduit of the respiratory therapy system. Thus, pressurized air is delivered indirectly from the conduit of the respiratory therapy system into the volume of space defined by the cushion (or the cushion and frame) of the user interface against the user's face. Moreover, according to some implementations, the indirectly connected category of user interfaces can be described as being at least two different categories: “indirect headgear” and “indirect conduit”. For the indirect headgear category, the conduit of the respiratory therapy system connects to a headgear conduit, optionally via a connector, which in turn connects to the cushion (or frame, or cushion and frame). The headgear is therefore configured to deliver the pressurized air from the conduit of the respiratory therapy system to the cushion (or frame, or cushion and frame) of the user interface. This headgear conduit within the headgear of the user interface is therefore configured to deliver the pressurized air from the conduit of the respiratory therapy system to the cushion of the user interface.

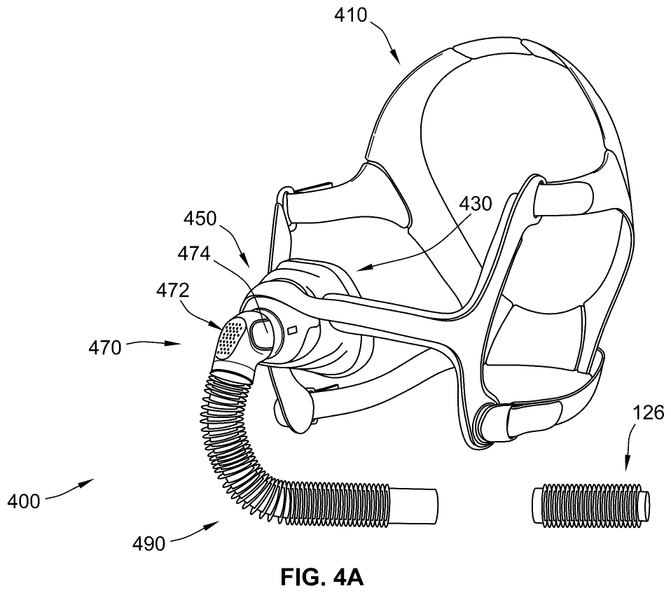

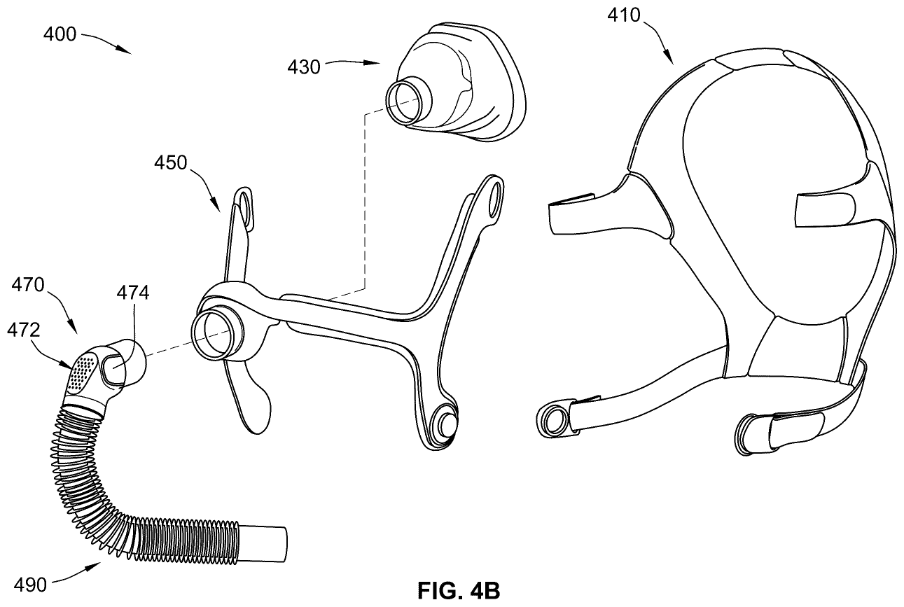

FIGS. 4A and 4B illustrate a perspective view and an exploded view, respectively, of one implementation of an indirect conduit user interface 400, according to aspects of the present disclosure. The indirect conduit user interface 400 includes a cushion 430 and a frame 450. In some embodiments, the cushion 430 and frame 450 form a unitary component of the user interface 400. The indirect conduit user interface 400 may further be considered to include a headgear 410, such as a strap assembly, a connector 470, and a user interface conduit 490 (often referred to in the art as a “minitube” or a “flexitube”).

Generally, the user interface conduit is (i) is more flexible than the conduit 126 of the respiratory therapy system, (ii) has a diameter smaller than the diameter of the than the than the conduit 126 of the respiratory therapy system, or both (i) and (ii). Similar to the headgear 310 of user interface 300, the headgear 410 of user interface 400 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The connector 470 is configured to couple to the frame 450 and/or cushion 430 at one end and to the conduit 490 of the user interface 400 at the other end. In other implementations, the conduit 490 may connect directly to frame 450 and/or cushion 430. The conduit 490, at the opposite end relative to the frame 450 and cushion 430, is configured to connect to the conduit 126 (FIG. 4A) of the respiratory therapy system (not shown). The pressurized air can flow from the conduit 126 (FIG. 4A) of the respiratory therapy system, through the user interface conduit 490, and the connector 470, and into a volume of space define by the cushion 430 (or cushion 430 and frame 450) of the user interface 400 against a user's face. From the volume of space, the pressurized air reaches the user's airway through the user's mouth, nose, or both.

In view of the above configuration, the user interface 400 is an indirectly connected user interface because pressurized air is delivered from the conduit 126 (FIG. 4A) of the respiratory therapy system (not shown) to the cushion 430 (or frame 450, or cushion 430 and frame 450) through the user interface conduit 490, rather than directly from the conduit 126 (FIG. 4A) of the respiratory therapy system.

As shown, in some implementations, the connector 470 includes a plurality of vents 472 for permitting the escape of carbon dioxide (CO₂) and other gases exhaled by the user when the respiratory therapy device is active. In some such implementations, each of the plurality of vents 472 is an opening that may be angled relative to the thickness of the connector wall through which the opening is formed. The angled openings can reduce noise of the CO₂ and other gases escaping to the atmosphere. Because of the reduced noise, acoustic signal associated with the plurality of vents 472 may be more apparent to an internal microphone, as opposed to an external microphone.

In some implementations, the connector 470 optionally includes at least one valve 474 for permitting the escape of CO₂ and other gases exhaled by the user when the respiratory therapy device is inactive. In some implementations, the valve 474 (an example of an anti-asphyxia valve) includes a silicone flap that is a failsafe component, which allows CO₂ and other gases exhaled by the user to escape in the event that the vents 472 fail when the respiratory therapy device is active. In some such implementations, when the silicone flap is open, the valve opening is much greater than each vent opening, and therefore less likely to be blocked by occlusion materials.

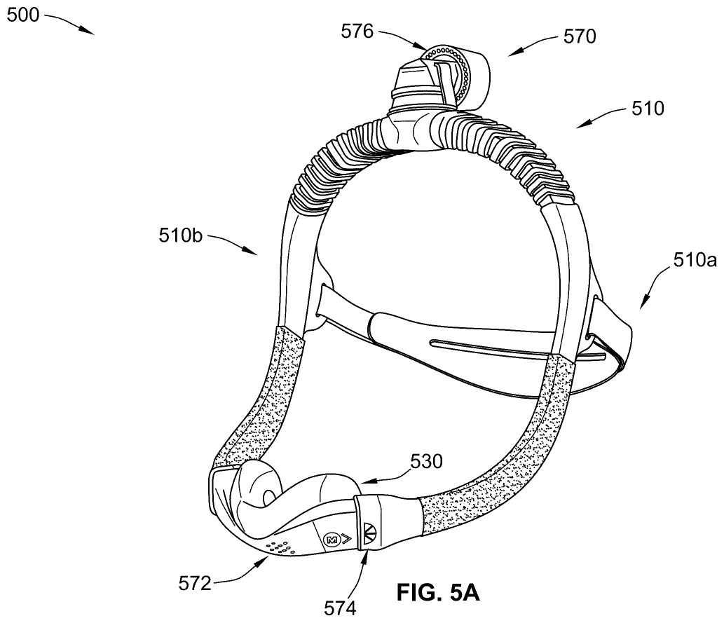

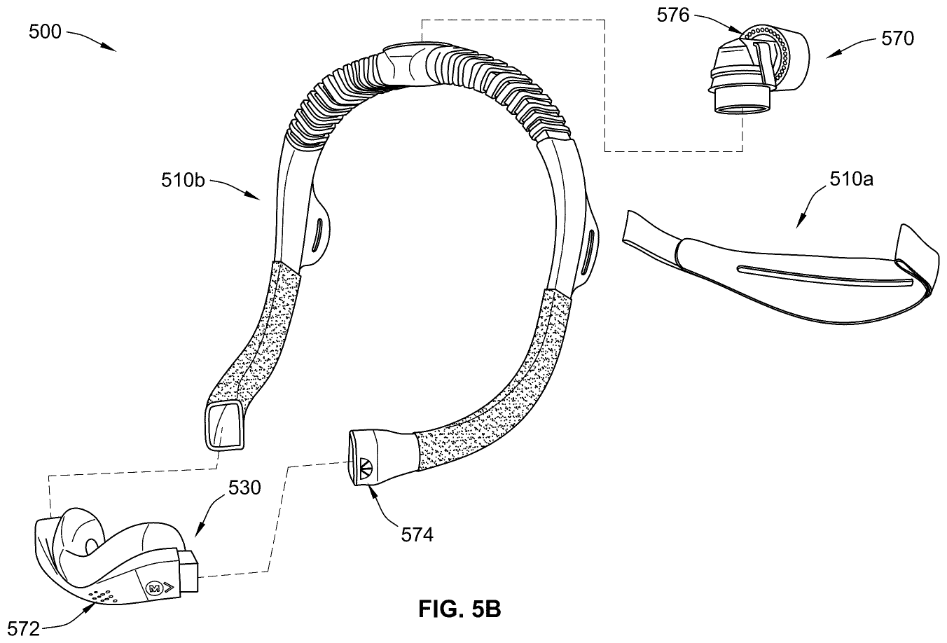

FIGS. 5A and 5B illustrate a perspective view and an exploded view, respectively, of one implementation of an indirect headgear user interface 500, according to aspects of the present disclosure. The indirect headgear user interface 500 includes a cushion 530. The indirect headgear user interface 500 may further be considered to comprise headgear 510 (which can comprise strap 510a and a headgear conduit 510b, and a connector 570. Similar to the user interfaces 300 and 400, the headgear 510 is configured to be positioned generally about at least a portion of a user's head when the user wears the user interface 500. The headgear 510 includes a strap 510a that can be coupled to the headgear conduit 510b and positioned on the user's head such that the user's head is positioned between the strap 510a and the headgear conduit 510b. The cushion 530 is positioned between the user's face and the headgear conduit 510b to form a seal on the user's face. The connector 570 is configured to couple to the headgear 510 at one end and a conduit of the respiratory therapy system at the other end. In other implementations, the connector 570 can be optional and the headgear 510 can alternatively connect directly to conduit of the respiratory therapy system. The headgear conduit 510b may be configured to deliver pressurized air from the conduit of the respiratory therapy system to the cushion 530, or more specifically, to the volume of space around the mouth and/or nose of the user and enclosed by the user cushion. Thus, the headgear conduit 510b is hollow to provide a passageway for the pressurized air. Both sides of the headgear conduit 510b can be hollow to provide two passageways for the pressurized air. Alternatively, only one side of the headgear conduit 510b can be hollow to provide a single passageway. In the implementation illustrated in FIGS. 5A and 5B, headgear conduit 510b comprises two passageways which, in use, are positioned at either side of a user's head/face. Alternatively, only one passageway of the headgear conduit 510b can be hollow to provide a single passageway. The pressurized air can flow from the conduit of the respiratory therapy system, through the connector 570 and the headgear conduit 510b, and into the volume of space between the cushion 530 and the user's face. From the volume of space between the cushion 530 and the user's face, the pressurized air reaches the user's airway through the user's mouth, nose, or both.

In some implementations, the cushion 530 may include a plurality of vents 572 on the cushion 530 itself. Additionally or alternatively, in some implementations, the connector 570 may include a plurality of vents 576 (“diffuser vents”) in proximity to the headgear 510, for permitting the escape of carbon dioxide (CO₂) and other gases exhaled by the user when the respiratory therapy device is active. In some implementations, the headgear 510 may include at least one plus anti-asphyxia valve (AAV) 574 in proximity to the cushion 530, which allows CO₂ and other gases exhaled by the user to escape in the event that the vents (e.g., the vents 572 or 576) fail when the respiratory therapy device is active.

In view of the above configuration, the user interface 500 is an indirect headgear user interface because pressurized air is delivered from the conduit of the respiratory therapy system to the volume of space between the cushion 530 and the user's face through the headgear conduit 510b, rather than directly from the conduit of the respiratory therapy system to the volume of space between the cushion 530 and the user's face.

In one or more implementations, the distinction between the direct category and the indirect category can be defined in terms of a distance the pressurized air travels after leaving the conduit of the respiratory therapy device and before reaching the volume of space defined by the cushion of the user interface forming a seal with the user's face, exclusive of a connector of the user interface that connects to the conduit. This distance is shorter, such as less than 1 centimeter (cm), less than 2 cm, less than 3 cm, less than 4 cm, or less than 5 cm, for direct category user interfaces than for indirect category user interfaces. This is because the pressurized air travels through the additional element of, for example, the user interface conduit 490 or the headgear conduit 510b between the conduit of the respiratory therapy system before reaching the volume of space defined by the cushion (or cushion and frame) of the user interface forming a seal with the user's face for indirect category user interfaces.

Referring back to FIG. 1, the conduit 126 (also referred to as an air circuit or tube) allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

Referring briefly to FIG. 6, a perspective view of the back side of the respiratory therapy device 122 that includes a housing 123, an air inlet 186, and an air outlet 190. The air inlet 186 includes an inlet cover 182 movable between a closed position and an open position. The air inlet cover 182 includes one or more air inlet apertures 184 defined therein. The respiratory therapy device 122 includes a blower motor configured to draw air in through the one or more air inlet apertures 184 defined in the air inlet cover 182. The motor is further configured to cause pressurized air to flow through the humidification tank 129 and out of the air outlet 190. The conduit 126 can be fluidly coupled to the air outlet 190, such that the air flows from the air outlet 190 and into the conduit 126. The air outlet 190 is partially formed by an internal conduit 192 extending through the housing 123 from the interior of the respiratory therapy device 122. A seal 194 is positioned around the end of the internal conduit 192 to ensure that substantially all of the air that exits through the air outlet 190 flows into the conduit 126.

Referring back to FIG. 1, the display device 128 is generally used to display image(s) including still images, video images, or both and/or information regarding the respiratory therapy device 122. For example, the display device 128 (and/or the display device 172 of the user device 170) can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in WO 2016/061629, which is hereby incorporated by reference herein in its entirety; the current date/time; personal information for the user 210; etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (also referred to herein as a mask, e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, acoustic data, or both. Physiological data generated by one or more of the sensors 130 can be used by the control system 110 to determine a sleep-wake signal associated with the user 210 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, WO 2014/047310, US 2014/0088373, WO 2017/132726, WO 2019/122413, and WO 2019/122414, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or acoustic data generated by the one or more sensors 130 can also be used to determine a respiration signal associated with a user during a sleep session. The respiration signal is generally indicative of respiration or breathing of the user during the sleep session. The respiration signal can be indicative of and/or analyzed to determine (e.g., using the control system 110) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 122, or any combination thereof. The one or more events can include snoring, apneas, central apneas, obstructive apneas, mixed apneas, hypopneas, a mask leak (e.g., from the user interface 124), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 130, or from other types of data.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in WO 2012/012835, which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 can be located at any location relative to the respiratory therapy system 120 and in acoustic communication with the airflow in the respiratory therapy system 120. For example, the respiratory therapy system 120 may include a microphone 140 (i) coupled externally to the conduit 126, (ii) positioned within, optionally at least partially within the respiratory therapy device 122, (iii) coupled externally to the user interface 124, (iv) coupled directly or indirectly to a headgear associated with the user interface 124, or in any other suitable location. In some implementations, the microphone 140 is coupled to a mobile device (for example, the user device 170 or a smart speaker(s) such as Google Home, Amazon Echo, Alexa etc.) that is communicatively coupled to the respiratory therapy system 120.

In some implementations, the microphone 140 is positioned on or at least partially outside of a housing of the respiratory therapy device 122. For example, the microphone 140 may be at least partially movable relative to the housing of the respiratory therapy device 122 to aid in being directed to the user 210 (FIG. 2). For example, the microphone 340 can be rotated between about 5° and about 355° towards the user 210.

In some implementations, the microphone 140 is configured to be in direct fluid communication with the airflow in the respiratory therapy system 120. For example, the microphone 140 may be (i) positioned at least partially within the conduit 126, (ii) positioned at least partially within the respiratory therapy device 122, optionally positioned at least partially within a component of the respiratory therapy device 122, which is in fluid communication with the conduit 126, or (iii) positioned at least partially within the user interface 124, the user interface 124 being in fluid communication with the conduit 126. Further, in some implementations, the microphone 140 is electrically connected with a circuit board (for example, connected physically, such as mounted on, the circuit board directly or indirectly) of the respiratory therapy device 122, which may be in acoustic communication (for example, via a small duct and/or a silicone window as in a stethoscope) or in fluid communication with the airflow in the respiratory therapy system 120.

The microphone 140 outputs sound and/or acoustic data that can be stored in the memory device 114 and/or analyzed by the processor 112 of the control system 110. The acoustic data generated by the microphone 140 is reproducible as one or more sound(s) during a sleep session (e.g., sounds from the user 210). The acoustic data form the microphone 140 can also be used to identify (e.g., using the control system 110) an event experienced by the user during the sleep session, as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the acoustic data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a SONAR sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a SONAR sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, or any combination thereof. For example, the microphone 140 and the speaker 142 can be integrated in and/or coupled to the user device 170 and the pressure sensor 132 and/or flow rate sensor 134 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory device 114, the respiratory therapy system 120, and/or the user device 170.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Generally, the sleep session includes any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake.

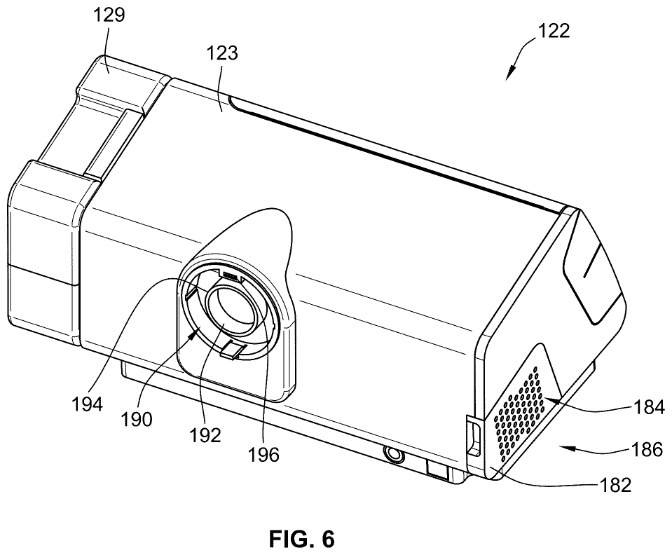

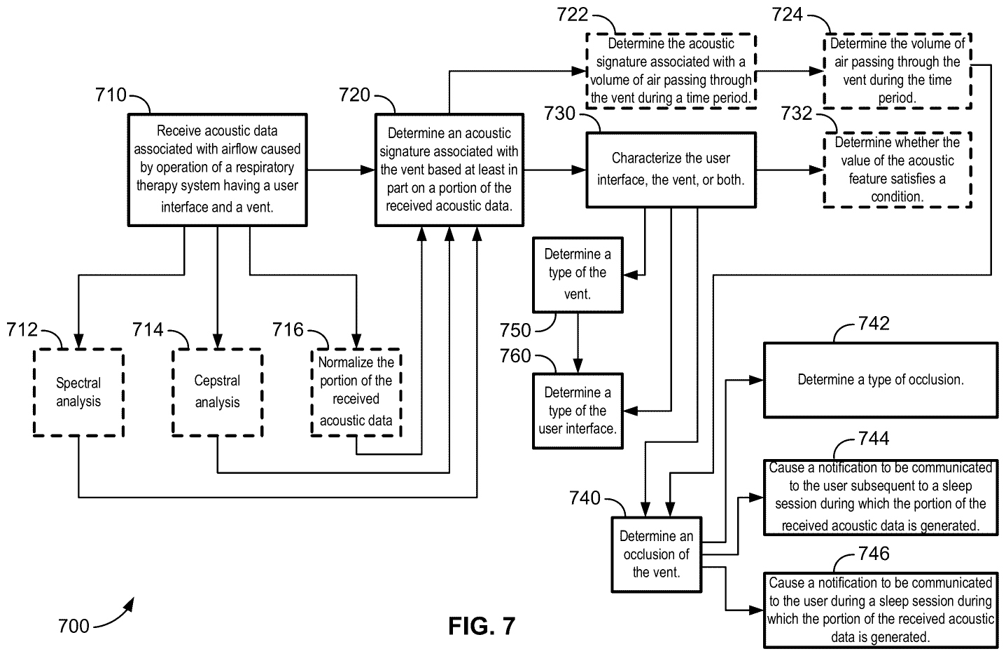

Referring to FIG. 7, a method 700 for characterizing a user interface (e.g., the user interface 124 of the system 100) and/or a vent of the user interface according to some implementations of the present disclosure is illustrated. One or more steps of the method 700 can be implemented using any element or aspect of the system 100 (FIGS. 1-2) described herein. While the method 700 has been shown and described herein as occurring in a certain order, more generally, the steps of the method 700 can be performed in any suitable order.

The method 700 provides, at step 710, acoustic data associated with airflow caused by operation of a respiratory therapy system (e.g., the respiratory therapy system 120 of FIGS. 1-2) is received. The respiratory therapy system is configured to supply pressurized air to a user (e.g., the user 210 of FIG. 2). The respiratory therapy system includes a user interface and a vent. In some implementations, the user interface is configured to engage a face of the user and deliver the pressurized air to an airway of the user during a therapy session. As described herein, in some implementations, the respiratory therapy system can include more than one vent, which may be located in the user interface and/or a connector to the user interface.

In some implementations, the acoustic data received at step 710 is associated with and/or generated during (i) one or more prior sleep sessions of the user of the respiratory therapy system, (ii) a current sleep session of the user of the respiratory therapy system, (iii) a beginning of the current session of the user of the respiratory therapy system, (iv) one or more sleep sessions of one or more users of respiratory therapy systems, or (v) any combination thereof. In some such implementations, the beginning of the current session refers to the first 1-15 minutes of the current sleep session, such as the first one minute, two minutes, three minutes, five minutes, ten minutes, or 15 minutes of the current sleep session. Additionally or alternatively, in some such implementations, the beginning of the current session refers to the ramp phase of the sleep session.

In some implementations, the acoustic data received at step 710 is generated, at least in part, by one or more microphones (e.g., the microphone 140 of the system 100) communicatively coupled to the respiratory therapy system, such as described above with respect to the microphone 140. In preferred implementations, the one or more microphones are located within the respiratory therapy device 122 (and/or the conduit 126 or user interface 124) and in acoustic and/or fluid communication with the airflow in the respiratory therapy system 120, which location may provide greater acoustic sensitivity when detecting acoustic signals associated with passage of gas through the vent compared to, for example, an external microphone.

The method 700 further provides, at step 720, an acoustic signature associated with the vent is determined, based at least in part on a portion of the acoustic data received at step 710. For example, in some implementations, the acoustic signature determined at step 720 is indicative of a volume of air passing through the vent of the respiratory therapy system.

In some implementations, the vent is configured to permit escape of gas (e.g., the respired pressurized air) exhaled by the user of the respiratory therapy system. For example, the gas exhaled by the user may contain at least a portion of the pressurized air supplied to the user. The gas exhaled by the user may be permitted to escape to atmosphere and/or outside of the respiratory therapy system. In some implementations, the acoustic signature determined at step 720 is associated with sounds of the exhaled gas escaping from the vent.

In some implementations, the portion of the received acoustic data used to determine the acoustic signature at step 720 is generated during a breath of the user. The breath may include an inhalation portion and an exhalation portion. In some such implementations, the portion of the received acoustic data is generated at least at a first time, a second time, or both. The first time is within the inhalation portion of the breath, optionally about a beginning of the inhalation portion of the breath. The beginning of the inhalation portion of the breath is associated with a minimum flow volume value of the breath, where the flow volume value is associated with the pressurized air supplied to the user of the respiratory therapy system. The second time is within the exhalation portion of the breath, optionally about a beginning of the exhalation portion of the breath. In contrast to the beginning of the inhalation portion of the breath, the beginning of the exhalation portion of the breath is associated with a maximum flow volume value of the breath. By being generated about a beginning of the inhalation and/or exhalation portion of the breath, the acoustic data is generated at points in the breathing cycle of the user when confounding factors due to a user's breathing which may affect the quality of the acoustic data are minimized.

Additionally or alternatively, in some implementations, the portion of the received acoustic data used to determine the acoustic signature at step 720 is generated during a plurality of breaths of the user, where each breath includes an inhalation portion and an exhalation portion as described above.

In some implementations, the method 700 further includes a spectral analysis of the portion of the acoustic data at step 712. The acoustic signature is then determined, at step 720, based at least in part on the spectral analysis. For example, the spectral analysis may include (i) generation of a discrete Fourier transform (DFT), such as a fast Fourier transform (FFT), optionally with a sliding window; (ii) generation of a spectrogram; (iii) generation of a short time Fourier transform (STFT); (iv) a wavelet-based analysis; or (v) any combination thereof. In some implementations, the acoustic signatures associated with the vent (e.g., vent signatures) can be more stationary compared to other acoustic phenomena (such as snoring, speech, etc.). These vent signatures depend on the underlying pressure, leak, and/or whether the vent is blocked (which might occur, for example, during the night with the user changing body position). Thus, the method 700 is configured to (i) extract spectra (or other transforms, including cepstra) on segments of acoustic data where conditions can be assumed to be quasi-stationary, (ii) perform some averaging to remove transient effects (such as differences between inspiration and expiration), then (iii) normalize the resulting data to account for these slower scale changes (e.g. pressure). Additionally, in some implementations, acoustic data may be removed from analysis regions where there is strong intensity acoustic interference (e.g., from speech), which can be done based on time domain variability.

In some implementations, the method 700 further includes, in addition or as an alternative to step 712, a cepstral analysis of the portion of the acoustic data at step 714. The acoustic signature is then determined, at step 720, based at least in part on the cepstral analysis. For example, the cepstral analysis may include: generating a mel-frequency cepstrum from the portion of the received acoustic data; and determining one or more mel-frequency cepstral coefficients from the generated mel-frequency cepstrum. The acoustic signature then includes the one or more mel-frequency cepstral coefficients. In some implementations, the one or more mel-cepstral coefficients are examples of features that may be extracted from the cepstra. Similar steps may be performed, where mel-spectral coefficients are examples of features that may be extracted from the spectra.

In some implementations, the method 700 optionally further provides, at step 716, the portion of the acoustic data received at step 710 is normalized. For example, in some such implementations, a mean power in a frequency region (e.g., 9-10 k Hz, and/or where the spectrum settles) is calculated. Where the spectrum settles is likely to be correlated with the noise created by turbulence, and associated mainly with increase in flow rate and pressure. The spectrum can be divided by this value (e.g., the calculated mean power) instead of the mean across all frequencies ranges. In some implementations, the normalization (step 716) could be done after the spectra analysis (step 712) or cepstra analysis (step 714). In some such implementations, the normalizing the portion of the received acoustic data at step 716 accounts for confounding conditions, for example, attributable to microphone gain, breathing amplitude, therapy pressure, or any combination thereof. The acoustic signature is then determined, at step 720, after the portion of the acoustic data is normalized at step 716.

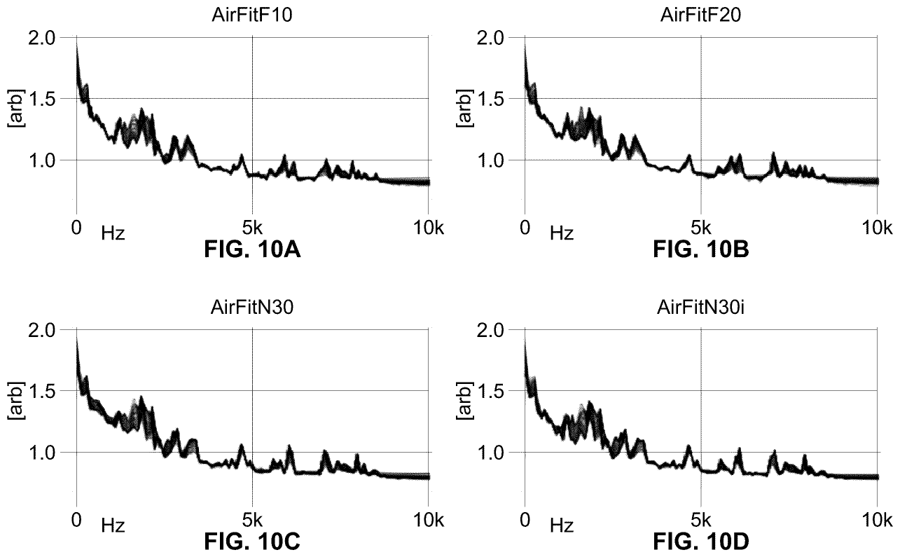

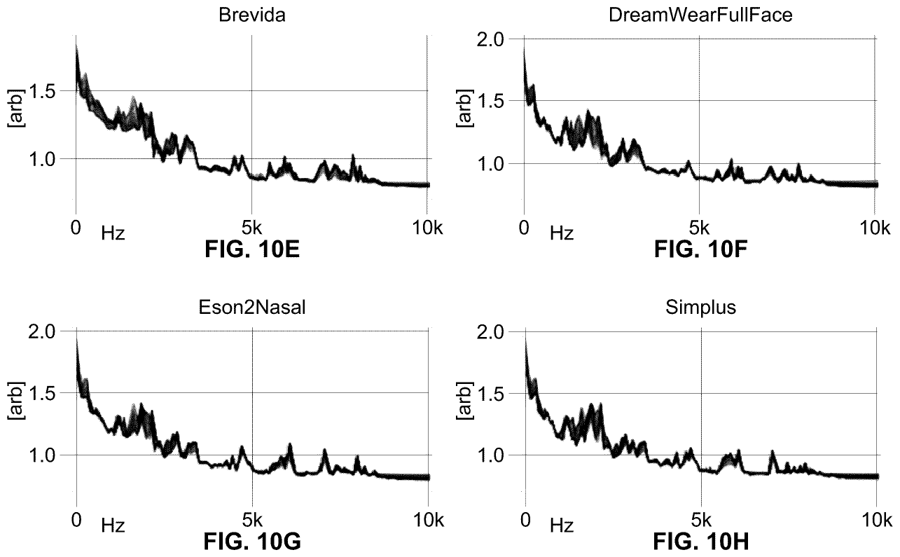

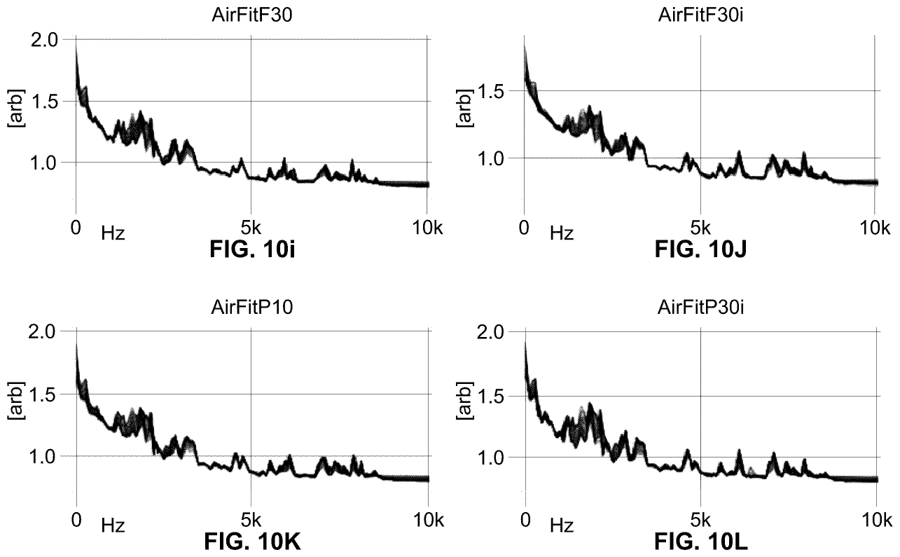

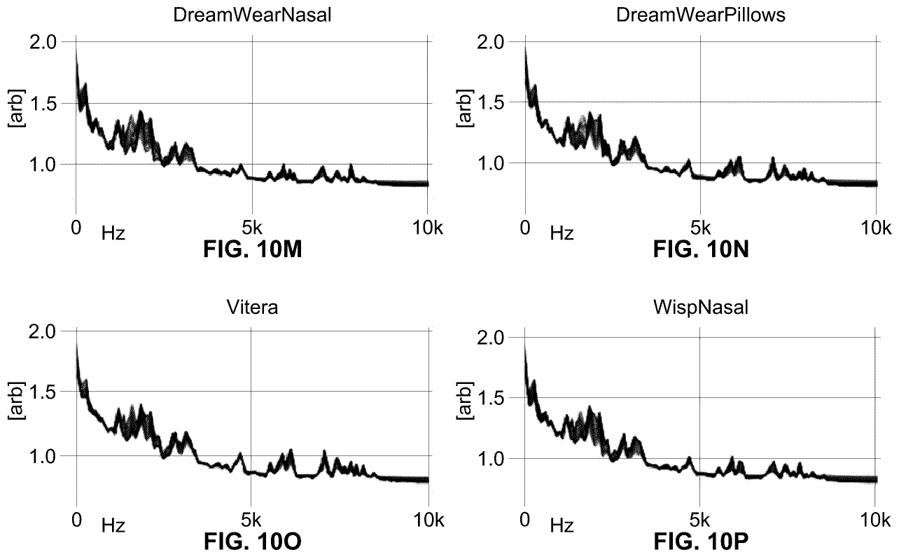

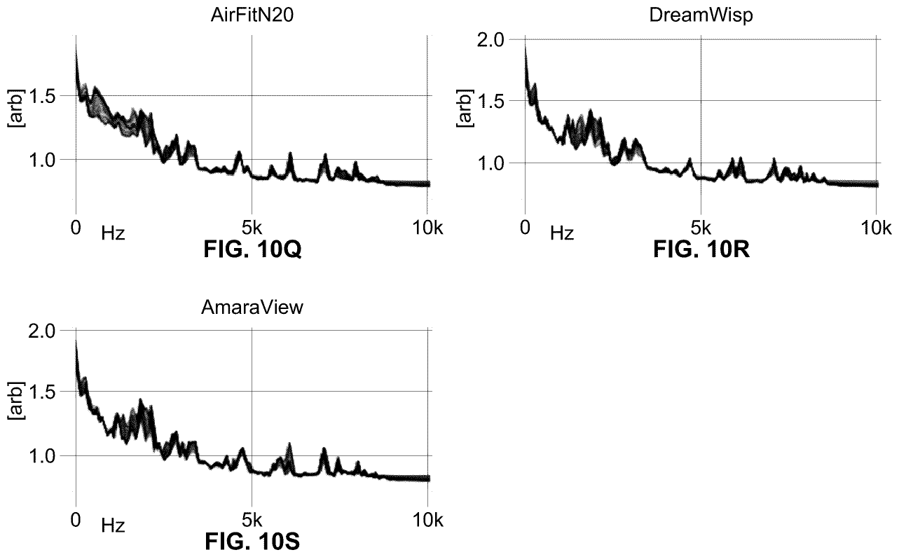

The method 700 further provides, at step 730, the user interface, the vent, or both are characterized, based at least in part on the acoustic signature associated with the vent determined at step 720. In some implementations, the vent type may be unique to a user interface and thus the acoustic signature associated with the vent can characterize the user interface. Additionally or alternatively, in some implementations, the combination of a non-unique vent with a user interface creates a unique acoustic signature from which the user interface can be characterized. Further additionally or alternatively, in some implementations: (i) for normal functioning vents (e.g., not occluded by debris, or actively against a pillow such as when the user moves during sleep), the acoustic signature(s) are aligned across different user interfaces of the same type, provided that different pressure conditions are taken into account (which can be normalized)—this may then become the baseline; (ii) for occluded vents, the acoustic signature(s) are different with respect to the baseline (e.g. the power in some frequency bands can get dampened, as exemplified in FIGS. 10A-10S herein); and (iii) a classifier may be constructed where we can train with data collected on various user interface types and with different levels of occlusion, which then would be able to both distinguish between different user interface types and degrees of occlusion.

For example, the user interface being characterized may include “direct category” user interfaces, “indirect category” user interfaces, direct/indirect headgear, direct/indirect conduit, or the like, such as the example types described with reference to FIGS. 3A-3B, 4A-4B, and 5A-5B. As another example, the user interface being characterized may include the following: AcuCare™ F1-0 non-vented (NV) full face mask, AcuCare™ F1-1 non-vented (NV) full face mask with AAV, AcuCare™ F1-4 vented full face mask, AcuCare™ high flow nasal cannula (HFNC), AirFit™ F10, AirFit™ F20, AirFit™ F30, AirFit™ F30i, AirFit™ masks for AirMini™, AirFit™ N10, AirFit™ N20, AirFit™ N30, AirFit™ N30i, AirFit™ P10, AirFit™ P30i, AirTouch™ F20, AirTouch™ N20, Mirage Activa™, Mirage Activa™ LT, Mirage™ FX, Mirage Kidsta™, Mirage Liberty™, Mirage Micro™, Mirage Micro™ for Kids, Mirage Quattro™, Mirage SoftGel™ Mirage Swift™ II, Mirage Vista™, Pixi™, Quattro™ Air, Quattro™ Air NV, Quattro™ FX, Quattro™ FX NV, ResMed© full face hospital mask, ResMed© full face hospital NV (non-vented) mask, ResMed© hospital nasal mask, Swift™ FX, Swift™ FX Bella, Swift™ FX Nano, Swift™ LT, Ultra Mirage™, Ultra Mirage™ II, Ultra Mirage™ NV (non-vented) full face mask, Ultra Mirage™ NV (non-vented) nasal mask, or any combination thereof.

In some implementations, the acoustic signature determined at step 720 includes an acoustic feature having a value. For example, the acoustic feature can include acoustic amplitude, acoustic volume, acoustic frequency, acoustic energy ratio, an energy content in a frequency band, a ratio of energy contents between different frequency bands, or any combination thereof. The value of the acoustic feature can include a maximum value, a minimum value, a range, a rate of change, a standard deviation, or any combination thereof.

In some such implementations, the characterizing at step 730 includes, at step 732, determining whether the value of the acoustic feature satisfies a condition. For example, the satisfying the condition includes exceeding a threshold value, not exceeding the threshold value, staying within a predetermined threshold range of values, or staying outside the predetermined threshold range of values. As another example, the satisfying the condition includes the combination of exceeding a threshold value and being within a predetermined threshold range of values.

The method 700 further provides, at step 740, an occlusion of the vent is determined. Additionally, in some implementations, in response to determining the occlusion of the vent at step 740, a type of occlusion is determined at step 742, based at least in part on the acoustic signature associated with the vent. The type of occlusion may correspond to full occlusion (e.g., 80%, 85%, 90%, 95% or more of the vents are occluded) or partial occlusion (e.g., less than full occlusion). Additionally or alternatively, the type of occlusion may correspond to sudden occlusion (e.g. due to rolling over on a pillow) or gradual occlusion (e.g. due to buildup of dirt), if the portion of the received acoustic data is generated over a time period (e.g. current data compared to, or combined with, historical/longitudinal data).

In some implementations, the determining the acoustic signature associated with the vent at step 720 further includes, at step 722, determining the acoustic signature associated with a volume of air passing through the vent during a time period. The occlusion of the vent is then associated with a reduced volume of air passing through the vent during the time period and a corresponding acoustic signature. For example, the determining the occlusion of the vent at step 720 may further include, at step 724, determining the volume of air passing through the vent during the time period (e.g., based on a value and duration of the acoustic signature).

In some implementations, the acoustic signature determined at step 720 includes changes relative to a baseline signature in one or more frequency bands. In some such implementations, the baseline signature can be associated with (i) a non-occluded vent, (ii) a vent with a known level of occlusion, and/or (iii) a vent with no active occlusion. For example, for active occlusion (e.g., occlusion that occurs due to the patient physically blocking the vent), the acoustic signature can include changes in the spectrum (or features extracted from that) along the sleep session of the user, thereby detecting when changes associated with blocking the vent might occur. For determining the occlusion at step 740, the one or more frequency bands may include (i) 0 kHz to 2.5 kHz, (ii) 2.5 kHz to 4 kHz, (iii) 4 kHz to 5.5 kHz, (iv) 5.5 kHz to 8.5 kHz, or (v) any combination thereof. The recited frequency bands and ranges are examples of suitable ranges based on the example user interfaces used herein, but other suitable frequency bands and ranges can be identified for other user interfaces. Additionally or alternatively, acoustic data associated with vents of additional user interfaces may be analyzed to determine specific signatures in different frequency bands, the union of which may be considered for an algorithm that would support all different types of user interfaces.

In some implementations, in response to determining the occlusion of the vent at step 740, a notification is caused to be communicated to the user or a third party at step 744, subsequent to a sleep session during which the portion of the received acoustic data is generated. Additionally or alternatively, in some implementations, in response to determining the occlusion of the vent at step 740, a notification is caused to be communicated to the user or a third party at step 746, during a sleep session during which the portion of the received acoustic data is generated. In some implementations, the third party includes a medical practitioner and/or a home medical equipment provider (HIME) for the user, who may understand (i) what user interface is used by and/or currently prescribed to the user, and/or (ii) how the current user interface is affecting the user in terms of therapy, leak, discomfort, etc.

For example, the notification at step 746 may be an alarm, a vibration, or via similar means, to wake or partially awaken the user, because a blocked vent may need to be remedied immediately, such as by having the user change head or body position. Additionally or alternatively, the notification may be send to a third party, such as a healthcare provider, user interface supplier or manufacturer, etc., which thus allows third party to take action if necessary, e.g. contact the user to suggest cleaning of a user interface or replacement of the user interface with the same or different type of user interface.

In some implementations, in response to determining the occlusion of the vent at step 740, a moisture sensor (e.g., the moisture sensor 176 of the system 100) is configured to determine an amount of condensation associated with the vent and/or the user interface. If the amount of condensation is higher than a baseline value of condensation, the respiratory therapy system may be configured to reduce an amount of moisture (e.g., via one or more settings associated with the humidification tank 129 of the system 100) being delivered via the airflow to the user.

In some implementations, the acoustic analysis can be used to distinguish a type of the user interface, with much of the acoustic signature being due to the vent. For example, the method 700 may further provide, at step 750, a type of the vent is determined based at least in part on the acoustic signature determined at step 720 and/or the characterization of the vent at step 730. In some implementations, the type of the vent determined at step 750 is indicative of a form factor of the user interface, a model of the user interface, a manufacturer of the user interface, a size of one or more elements of the user interface, or any combination thereof. In some implementations, the vent is located on a connector for a user interface that is configured to facilitate the airflow between the conduit of the respiratory therapy system and the user interface. In some such implementations, the type of the vent determined at step 750 is indicative of a form factor of the connector, a model of the connector, a manufacturer of the connector, a size of one or more elements of the connector, or any combination thereof.

In some implementations, the method 700 may further provide, at step 760, a type of the user interface is determined based at least in part on the acoustic signature determined at step 720 and/or the characterization of the user interfaced at step 730. For example, the type of user interface may include “direct category” user interfaces, “indirect category” user interfaces, direct/indirect headgear, direct/indirect conduit, or the like, such as the example types described with reference to FIGS. 3A-3B, 4A-4B, and 5A-5B. As a further example, the type of the user interface determined at step 760 includes a form factor of a user interface, a model of the user interface, a manufacturer of the user interface, a size of one or more elements of the user interface, or any combination thereof.

In some implementations, the acoustic signature determined at step 720 includes changes relative to a baseline signature in one or more frequency bands. For determining the type of the user interface at step 760, the one or more frequency bands may include (i) 4.5 kHz to 5 kHz, (ii) 5.5 kHz to 6.5 kHz, (iii) 7 kHz to 8.5 kHz, (iv) any combination thereof.

In some implementations, the acoustic data received at step 710 is generated during a plurality of sleep sessions associated with the respiratory therapy system. In some such implementations, the acoustic signature is determined (e.g., step 720) for each of the plurality of sleep sessions. Based at least in part on the determined acoustic signature for each of the plurality of sleep sessions, a condition of the vent may be determined. In some other such implementations, the occlusion of the vent is determined (e.g., at step 742) for each of the plurality of sleep sessions. Based at least in part on the determined occlusion of the vent for each of the plurality of sleep sessions, the condition of the vent may be determined. For example, the condition of the vent can include vent deterioration, vent deformation, vent damage, or any combination thereof.

Acoustic Signatures with Fully Open Vent

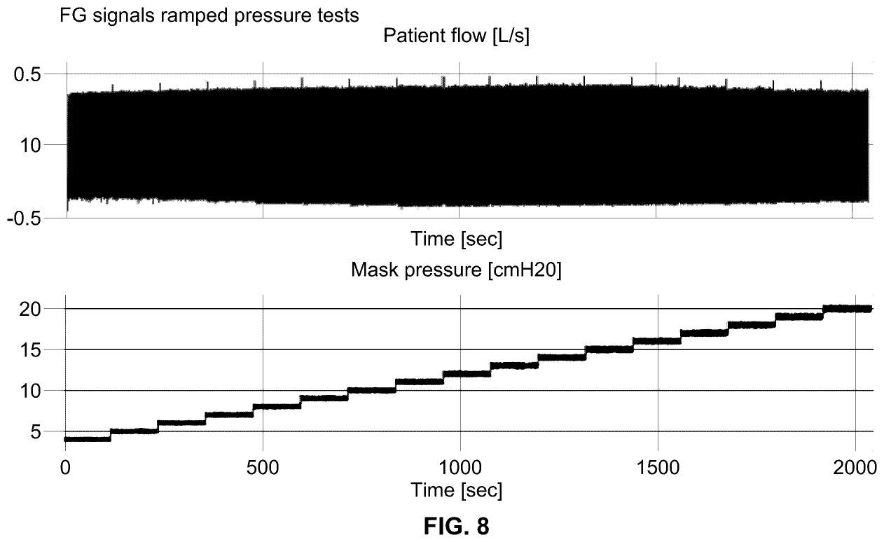

Controlled test data is generated using one or more steps of the method 700. In this example, acoustic data is collected with an internal microphone for a series of 19 user interfaces of their respective respiratory therapy systems, where the vents are fully open (i.e., not occluded). The pressure for each respiratory therapy system was ramped from 5 to 20 cmH₂O over a period of approximately 30 minutes, which is illustrated in FIG. 8. As shown, the patient flow varies approximately between −0.5 L/s to +0.5 L/s.

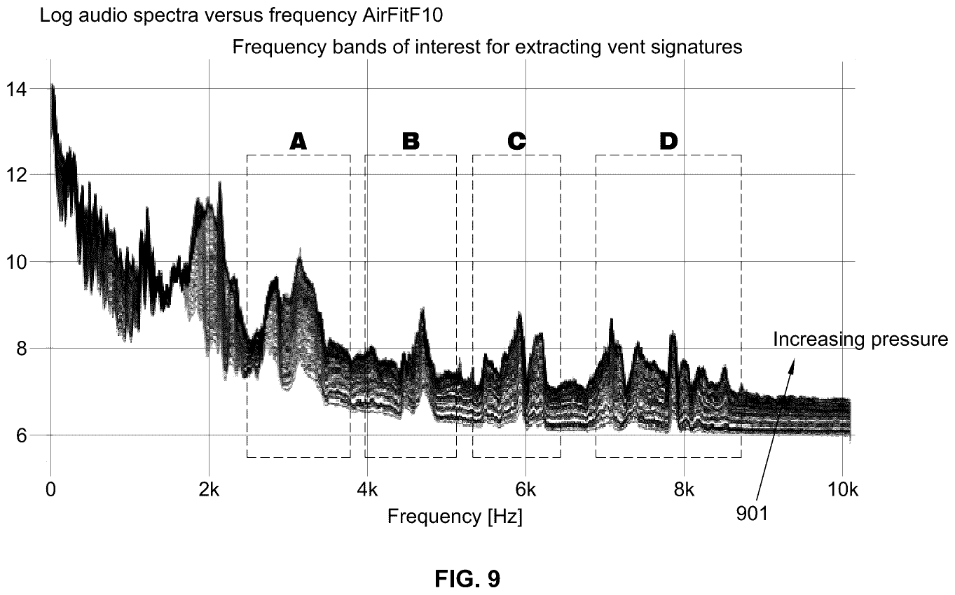

Referring to FIG. 9, the log audio spectra versus frequency during the pressure ramp-up of FIG. 8 is illustrated. In this example, an average spectrum was computed on each pressure step. As shown, the spectral signature of the acoustic data for this example user interface is dependent on the pressure. More specifically, the acoustic power increases with increased flow. The arrow 901 indicates increasing pressure, and the flow rate increases with increasing pressure. In this example, the frequency bands A (e.g., about 2.2 kHz-about 3.9 kHz), B (e.g., about 4.0 kHz-about 5.1 kHz), C (e.g., about 5.3 kHz-about 6.4 kHz), and D (e.g., about 6.8 kHz-about 8.6 kHz) are selected for extracting the acoustic signature associated with the vent. The recited frequency bands and ranges are examples of suitable ranges based on the example user interfaces used herein, but other suitable frequency bands and ranges can be identified for other user interfaces. Additionally or alternatively, acoustic data associated with vents of additional user interfaces may be analyzed to determine specific signatures in different frequency bands, the union of which may be considered for an algorithm that would support all different types of user interfaces.

As shown in FIG. 9, distinctive acoustic signature in the audible range can be seen for each mask type in these specific frequency bands, which is most likely associated with the vent flow, and background noise generated by the motor. In some implementations, environmental noise, breathing sounds, and/or speech would have time domain signatures (e.g., with increased variability), and could therefore be filtered out on that basis.

FIGS. 10A-10S illustrate the acoustic features based on normalized spectra (e.g., by dividing the spectrum by the mean power density) for each of the 19 user interfaces described above. These plots show the log of the audio spectral power density in the y-axis (represented by “[arb]”). As the acoustic data is digitized, it is proportional with de facto audio intensity of the actual source audio, provided that the microphone does not have an auto gain control mechanism. For example, this relation can be derived based on the microphone sensitivity. The acoustic features can include: (i) the number of peaks in each frequency band, (ii) the heights of the peaks, (iii) the widths of the peaks, (iv) the average power in each frequency band, (v) the ratio of the height of the peaks, or (vi) any combination thereof. Additionally or alternatively, in some implementations, other methods such as a 1D convolutional neural network may be used, which takes as input the entire spectra (e.g., all frequencies). As shown, when normalizing to eliminate the pressure dependencies, the spectra collapse, and the acoustic features differentiating different types of user interfaces can be seen in particular in the following frequency bands: (i) 4.5-5.0 kHz, (ii) 5.5-6.5 kHz, and (iii) 7.0-8.5 kHz. Regarding spectra collapse—without normalization, the magnitude of the spectra will be higher with increasing pressure (for each frequency). Thus, when normalization is performed, for each frequency, the magnitude of the spectra corresponding to different pressure is the same.

Acoustic Signatures with Partially or Fully Occluded Vent

Additional test data is generated using one or more steps of the method 700. In this example, a user interface (AirFitF20 model) is used with standard breathing profile from a breathing simulator Active Servo Lung (ASL), and the pressure setting at 10 cmH₂O. The test conditions include: (i) 5 minutes of fully open vent (i.e., not occluded), (ii) 5 minutes of partially occluded vent (i.e., diffuser only occluded), (iii) 5 minutes of fully occluded vent (i.e., diffuser only occluded), (iv) 5 minutes of complete occlusion (i.e., diffuser plus anti-asphyxia valve (AAV) outlet occluded). Spectra and cepstra are then calculated on 4,096 samples, which are sampled 0.1 second apart from one another, and averaged over 50 steps.

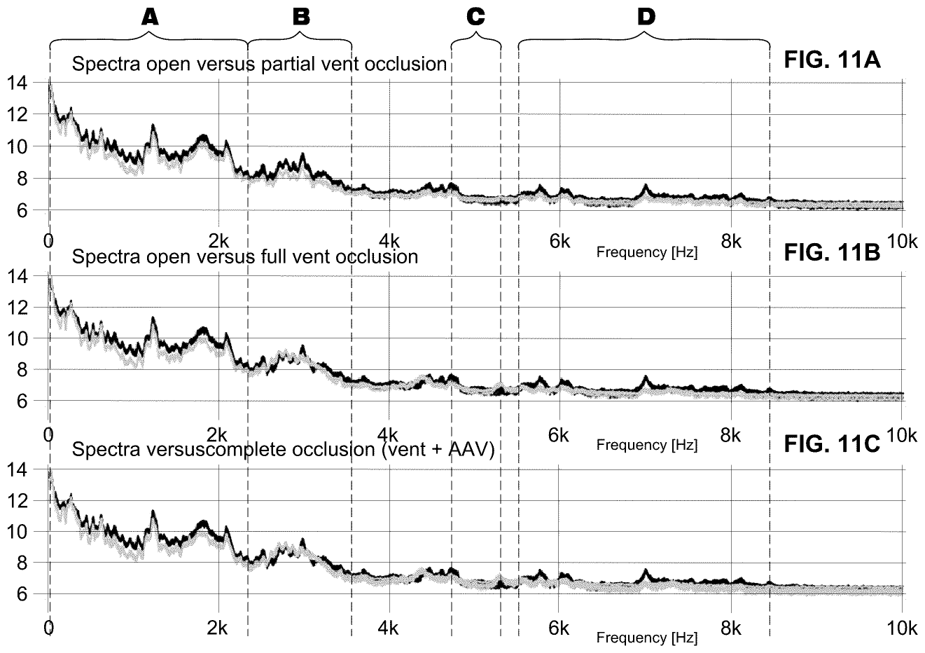

FIG. 11A illustrates the spectra acoustic signature versus frequency for open vents compared to partially occluded diffuser vents; FIG. 11B illustrates the spectra acoustic signature versus frequency for open vents compared to fully occluded diffuser vents; and FIG. 11C illustrates the spectra acoustic signature versus frequency for open vents compared to fully occluded diffuser vents plus AAV outlet. In other words, each figure compares spectra acoustic signature of the open vent (shown in black) versus occluded vent (shown in gray). Also, comparison across the spectra acoustic signatures allows partial occlusion versus full occlusion versus complete occlusion of vent and AAV to be distinguished.

As shown in FIGS. 11A-11C, changes in spectral signatures can be seen with respect to the baseline (e.g., open and not occluded vent), in particular in the following frequency bands: (i) 0 to 2.5 kHz (showing, at band A, a reduction in audio power for occluded vent versus fully open), (ii) 2.5 kHz to 4 kHz (showing, at band B, a reduction in audio power for partially occluded vent, and an increase in audio power for fully occluded vent), (iii) around 5.3 kHz (showing, at band C, a peak develops for fully occluded vent), and (iv) 5.5 kHz to 8.5 kHz (showing, at band D, a reduction in audio power for occluded vent versus fully open). Therefore, blockage and/or occlusion of the AAV outlet only has a small effect to the spectra acoustic signature across various frequency bands, partially because during therapy the AAV outlet has a very small amount of flow associated with it when the respiration therapy device is active.

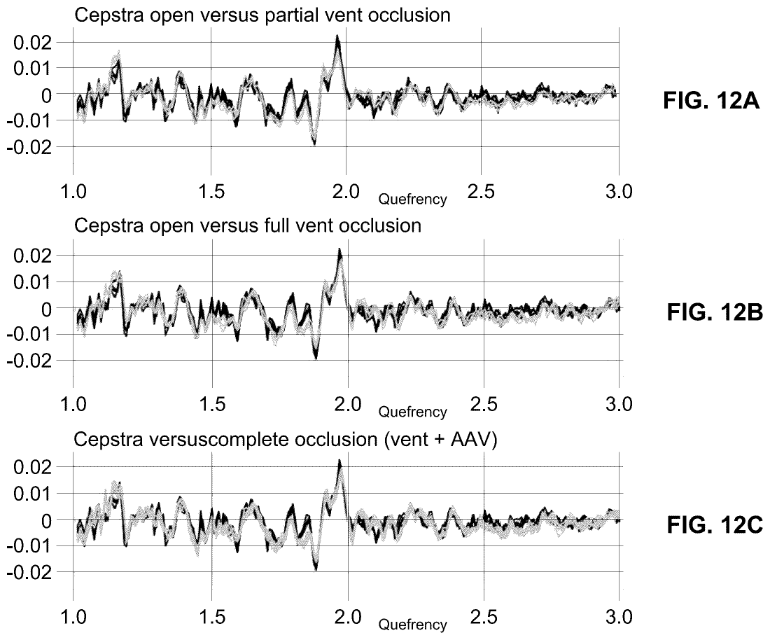

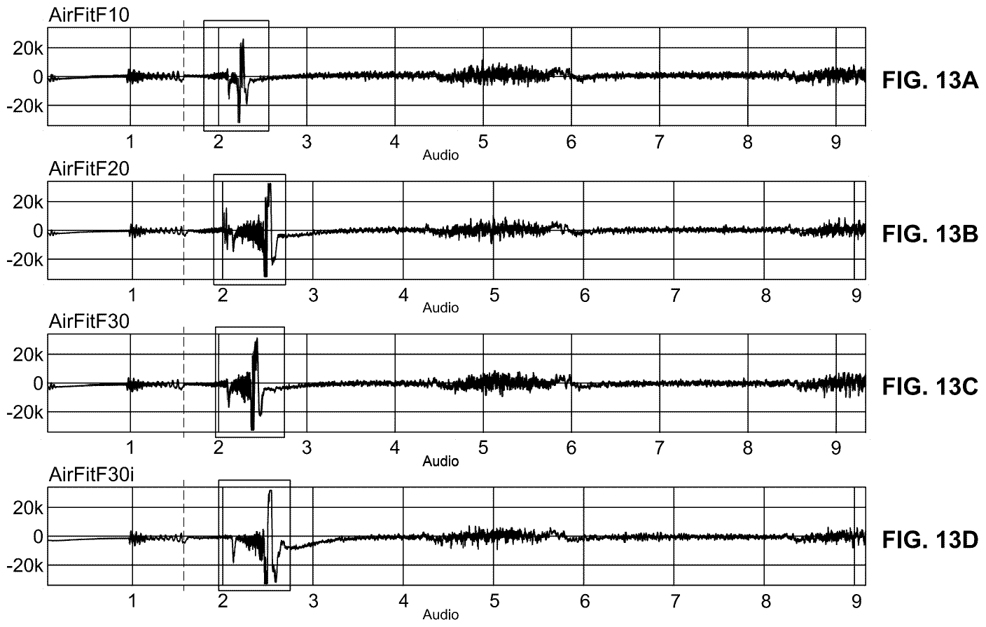

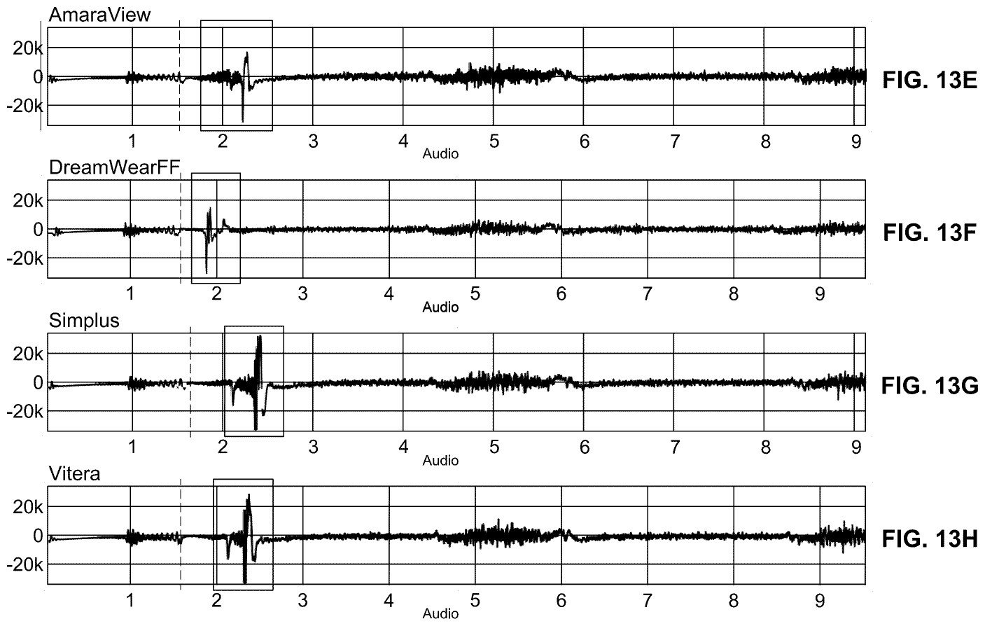

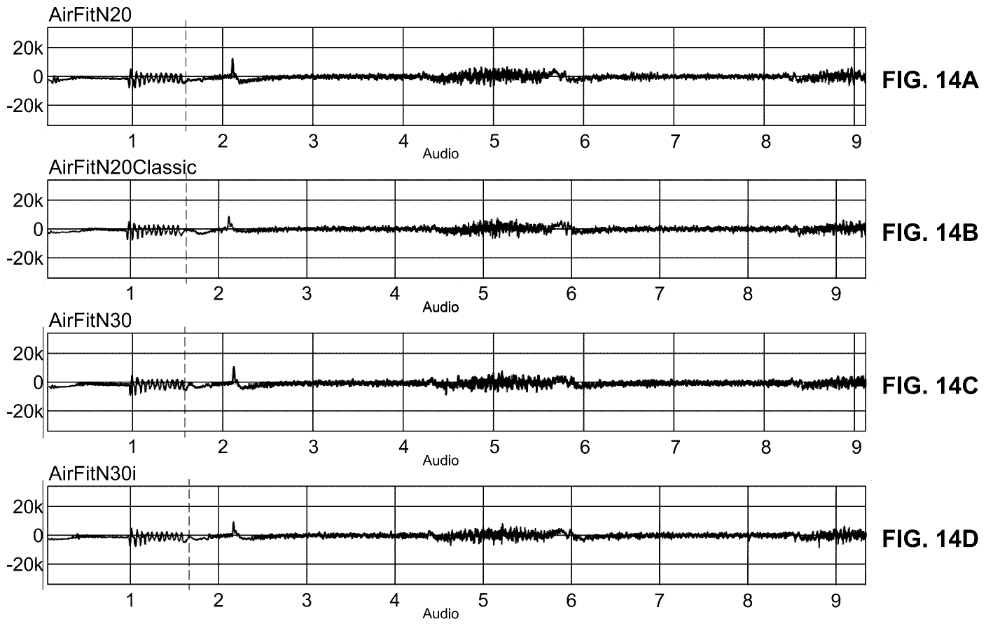

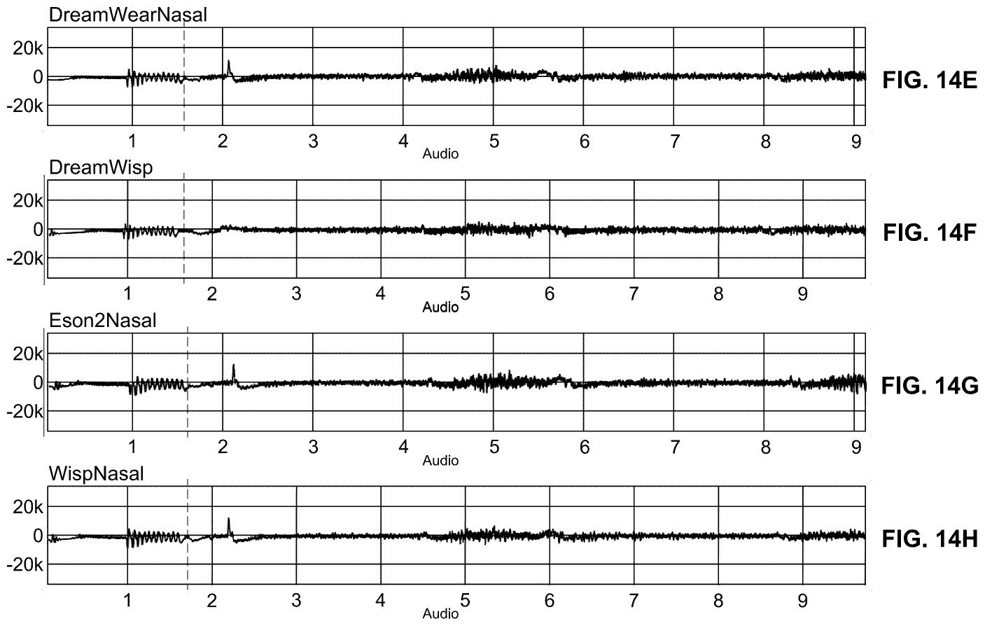

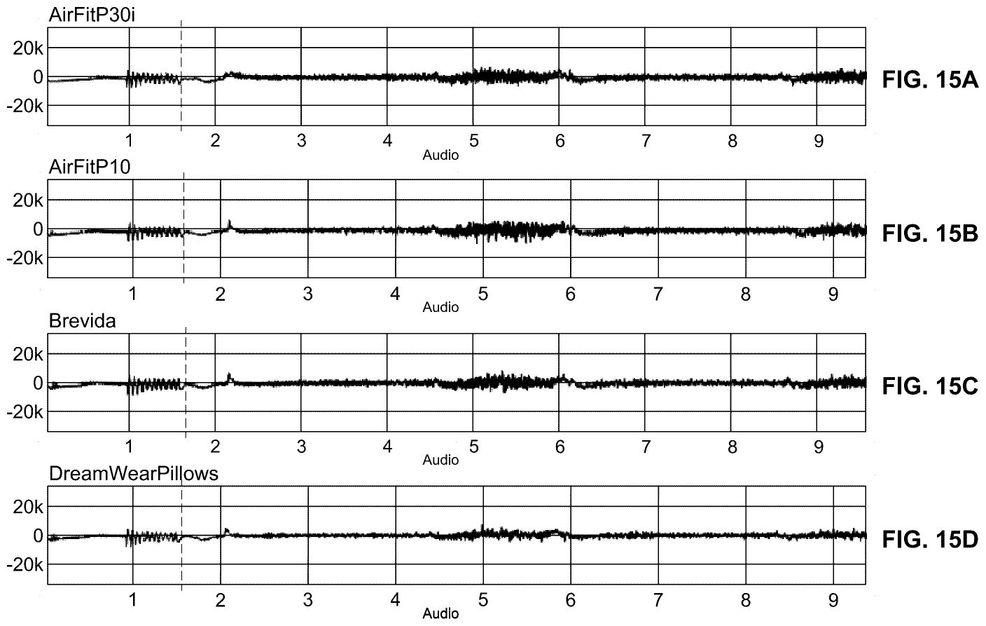

Referring now to FIGS. 12A-12C, cepstra analysis is performed similar to the spectral analysis referenced above with respect to FIGS. 11A-11C. More specifically, FIG. 12A illustrates the cepstra acoustic signature versus frequency for open vents compared to partially occluded diffuser vents; FIG. 12B illustrates the cepstra acoustic signature versus frequency for open vents compared to fully occluded diffuser vents; and FIG. 12C illustrates the cepstra acoustic signature versus frequency for open vents compared to fully occluded diffuser vents plus AAV outlet. In other words, each figure compares cepstra acoustic signature of the open vent (shown in black) versus occluded vent (shown in gray). As shown, vent occlusion has a smoothing effect on the cepstra, albeit small, and a comparison across the cepstra acoustic signatures allows partial occlusion versus full occlusion versus complete occlusion of vent and AAV to be distinguished. In some implementations, CO₂ build up may further impact the cepstra and help further differentiate occlusion events and/or occurrence.

One or more elements or aspects or steps, or any portion(s) thereof, from one or more of any of claims 1 to 60 below can be combined with one or more elements or aspects or steps, or any portion(s) thereof, from one or more of any of the other claims 1 to 60 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving acoustic data associated with airflow caused by operation of a respiratory therapy system configured to supply pressurized air to a user, the respiratory therapy system including a user interface and a vent;
determining, based at least in part on a portion of the received acoustic data, an acoustic signature associated with the vent; and
characterizing, based at least in part on the acoustic signature associated with the vent, the vent.

2. (canceled)

3. The method of claim 1, wherein the determined acoustic signature is indicative of a volume of air passing through the vent of the respiratory therapy system.

4. (canceled)

5. (canceled)

6. The method of claim 1 wherein the vent is configured to permit escape of gas exhaled by the user of the respiratory therapy system, and wherein the determined acoustic signature is associated with sounds of the exhaled gas escaping from the vent.

7. (canceled)

8. (canceled)

9. (canceled)

10. (canceled)

11. (canceled)

12. (canceled)

13. (canceled)

14. The method of claim 1, wherein the portion of the received acoustic data is generated during a breath of the user, and wherein the breath includes an inhalation portion and an exhalation portion, wherein the portion of the received acoustic data is generated at least at a first time, a second time, or both, wherein the first time is about a beginning of the inhalation portion of the breath, and wherein the beginning of the inhalation portion of the breath is associated with a minimum flow volume value of the breath, the flow volume being associated with the pressurized air supplied to the user of the respiratory therapy system.

15. (canceled)

16. (canceled)

17. (canceled)

18. (canceled)

19. (canceled)

20. (canceled)

21. (canceled)

22. (canceled)

23. (canceled)

24. (canceled)

25. The method of claim 1, wherein the determining the acoustic signature includes a cepstral analysis of the portion of the acoustic data, and wherein the acoustic signature is determined based at least in part on the cepstral analysis, wherein the cepstral analysis includes: generating a mel-frequency cepstrum from the portion of the received acoustic data; and determining one or more mel-frequency cepstral coefficients (MFCC) from the generated mel-frequency cepstrum, and wherein the acoustic signature includes the one or more MFCCs.

26. (canceled)

27. (canceled)

28. (canceled)

29. The method of claim 1, further comprising normalizing the portion of the received acoustic data, wherein the normalizing the portion of the received acoustic data includes (i) dividing a spectrum of the portion of the received acoustic data by mean power density, (ii) matching high frequency power level, or (iii) both (i) and (ii).

30. (canceled)

31. (canceled)

32. (canceled)

33. (canceled)

34. The method of claim 1, wherein the acoustic signature includes an acoustic feature having a value, and wherein the characterizing includes determining whether the value of the acoustic feature satisfies a condition, wherein the satisfying the condition includes exceeding a threshold value, not exceeding the threshold value, staying within a predetermined threshold range of values, or staying outside the predetermined threshold range of values.

35. (canceled)

36. (canceled)

37. (canceled)

38. The method of claim 1, wherein the characterizing includes determining a presence or absence of an anti-asphyxia valve.

39. The method of claim 38, wherein the acoustic signature used to determine the presence or absence of the anti-asphyxia valve includes an acoustic waveform detectable within about one to ten seconds, optionally one to three seconds, of initiation of air flow ramping phase using the respiratory therapy system.

40. (canceled)

41. (canceled)

42. (canceled)

43. (canceled)

44. The method of claim 1, wherein the characterizing includes determining an occlusion of the vent.

45. The method of claim 44, wherein the determining the acoustic signature associated with the vent includes determining the acoustic signature associated with a volume of air passing through the vent during a time period.

46. (canceled)

47. (canceled)

48. The method of claim 44, wherein the determined acoustic signature includes changes relative to a baseline signature in one or more frequency bands.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 1, wherein the acoustic data is generated during a plurality of sleep sessions associated with the respiratory therapy system, and wherein the method further comprises:
determining the acoustic signature for each of the plurality of sleep sessions; and
determining, based at least in part on the determined acoustic signature for each of the plurality of sleep sessions, a condition of the vent.

61. (canceled)

62. (canceled)

63. (canceled)

64. (canceled)

65. (canceled)

66. (canceled)

67. (canceled)

68. (canceled)

69. (canceled)

70. A system comprising:
a control system comprising one or more processors; and
a memory having stored thereon machine readable instructions which, when executed by the one or more processors, causes the control system to:
receive acoustic data associated with airflow caused by operation of a respiratory therapy system configured to supply pressurized air to a user, the respiratory therapy system including a user interface and a vent;
determine, based at least in part on a portion of the received acoustic data, an acoustic signature associated with the vent; and
characterize, based at least in part on the acoustic signature associated with the vent, the vent.

71. The system of claim 70, wherein the portion of the received acoustic data is generated during a breath of the user, and wherein the breath includes an inhalation portion and an exhalation portion, wherein the portion of the received acoustic data is generated at least at a first time, a second time, or both, wherein the first time is about a beginning of the inhalation portion of the breath, and wherein the beginning of the inhalation portion of the breath is associated with a minimum flow volume value of the breath, the flow volume being associated with the pressurized air supplied to the user of the respiratory therapy system.

72. The system of claim 70, wherein the determining the acoustic signature includes a cepstral analysis of the portion of the acoustic data, and wherein the acoustic signature is determined based at least in part on the cepstral analysis, wherein the cepstral analysis includes: generating a mel-frequency cepstrum from the portion of the received acoustic data; and determining one or more mel-frequency cepstral coefficients (MFCC) from the generated mel-frequency cepstrum, and wherein the acoustic signature includes the one or more MFCCs.

73. The system of claim 70, further comprising normalizing the portion of the received acoustic data, wherein the normalizing the portion of the received acoustic data includes (i) dividing a spectrum of the portion of the received acoustic data by mean power density, (ii) matching high frequency power level, or (iii) both (i) and (ii).

74. The system of claim 70, wherein the characterizing includes determining a presence or absence of an anti-asphyxia valve.

75. The system of claim 70, wherein the characterizing includes determining an occlusion of the vent.

76. The system of claim 70, wherein the acoustic data is generated during a plurality of sleep sessions associated with the respiratory therapy system, and wherein the method further comprises:
determining the acoustic signature for each of the plurality of sleep sessions; and
determining, based at least in part on the determined acoustic signature for each of the plurality of sleep sessions, a condition of the vent.
